(19) United States
(12) Patent Application Publication
(10) Pub. No.: US 2026/0256857 A1
(43) Pub. Date: Sep. 3, 2026

(54) METHODS AND COMPOSITIONS FOR TREATING OBESITY USING MICROORGANISMS

(71) Applicant: Microbiota I – Center (Magic) Limited, Shatin, N.T., Hong Kong SAR (CN)

(72) Inventors: Siew Chien NG, Hong Kong SAR (CN); Ka Leung Francis CHAN, Tai Po, N.T., Hong Kong SAR (CN); Zhan HUI, Tai Po, N.T., Hong Kong SAR (CN)

(21) Appl. No.: 18/878,863

(22) PCT Filed: Jun. 28, 2023
(86) PCT No.: PCT/CN2023/103144

§ 371 Date: Dec. 24, 2024

Related U.S. Application Data
(60) Provisional application No. 63/356,945, filed on Jun. 29, 2022.

(51) Int. Cl. A61K 36/07; A61K 36/062; A61K 36/064; A61P 3/04

(52) U.S. Cl. CPC A61K 36/07; A61K 36/062; A61K 36/064; A61P 3/04

(57) ABSTRACT

Compositions and methods for treating or reducing risk for developing obesity as well as associated diseases and disorders.

### RELATED APPLICATIONS

This application claims priority to U.S. Provisional Patent Application No. 63/356,945, filed Jun. 29, 2022, the contents of which are hereby incorporated in the entirety for all purposes.

### BACKGROUND OF THE INVENTION

As the living standards continue to improve globally, the number of individuals suffering from unhealthy physical conditions such as obesity and related metabolic diseases (especially type 2 diabetes) is also rapidly increasing. The World Health Organization (WHO) estimates that by 2030 the number of people living with diabetes will exceed 350 million worldwide. Due to the rising incidence of obesity, diabetes, and other metabolic diseases, their serious health implications, as well as their profound economic consequences, there exists an urgent need for new and effective means to treat high risk individuals, especially those who are already exhibiting early signs of high likelihood of disease development in the future, such as obesity, higher than normal blood cholesterol or triglyceride level, including higher than normal blood level of low-density lipoprotein cholesterol (LDL-C) and/or lower than normal blood level of high-density lipoprotein cholesterol (HDL-C), so as to reduce or eliminate their risk of later suffering from type 2 diabetes and other associated disorders, e.g., hypertension and cardiovascular disease.

The human gastrointestinal tract is host to a vast diversity of microbes, collectively known as the gut mycobiome. The compositions of various microbes in the gut mycobiome can be used as an indication of a person's general health conditions, as well as a person's risk or predisposition to developing certain diseases, such as metabolic diseases. Hence, the largest variety of microbes can serve as biomarkers and/or therapeutics for treating certain diseases.

There exists an urgent need for new and effective means to treat or prevent conditions among individuals who already have or might later develop diabetic and/or other metabolic diseases, so that prophylactic or therapeutic measures can be taken to delay the onset, reverse, or eliminate the symptoms of these diseases in these individuals or to reduce severity of the pertinent symptoms/risks associated with these diseases. The present invention fulfills this and other related needs by identifying beneficial gut microorganisms in an effort to formulate new compositions and devise new methods that are effective for improving metabolic health of individuals, especially among those at risk or already suffering from symptoms of various metabolic diseases related to obesity or diabetes.

### BRIEF SUMMARY OF THE INVENTION

The present inventors discovered in their studies the certain gut microbial species and their relative abundance can influence the metabolism of the host individual, leading to distinct effects on the individual's weight and metabolic health. Their findings provide an important utility in the prevention and treatment of diseases and conditions related to obesity and metabolic syndrome. The microorganisms so identified now serve to provide new methods and compositions for treating or preventing obesity and related conditions.

In a first aspect, the present invention provides a composition that is useful for preventing or treating obesity and related conditions in a human subject, for example, metabolic syndrome, type 2 diabetes, fatty liver, hypertension, cardiovascular disease etc. The composition contains an effective amount of (1) one or more of Schizosaccharomyces pombe, Cryptococcus neoformans, and Saccharomyces cerevisae; or an inhibitor of one or more of Lachancea thermotolerans, Saccharomyces paradoxus, Thielavia terrestris, Parastagonospora nodorum, and Myceliophthora thermophila, and (2) one or more physiologically acceptable excipients. In some embodiments, the composition consists essentially of or consists of the above specified components such as (1) one or more of Schizosaccharomyces pombe, Cryptococcus neoformans, and Saccharomyces cerevisae; or an inhibitor of one or more of Lachancea thermotolerans, Saccharomyces paradoxus, Thielavia terrestris, Parastagonospora nodorum, and Myceliophthora thermophila, and (2) one or more physiologically acceptable excipients. In some embodiments, the composition contains an effective amount of (1) Schizosaccharomyces pombe and/or Cryptococcus neoformans; or (2) an inhibitor of one or more of Lachancea thermotolerans, Saccharomyces paradoxus, and Thielavia terrestris. In some embodiments, the composition comprises an effective amount of Schizosaccharomyces pombe and/or Cryptococcus neoformans plus one or more physiological acceptable excipients. In some embodiments, the composition further includes at least one prebiotic, e.g., inulin and/or psyllium husk. In some embodiments, the composition is in the form of a food item or a beverage, which may be taken before, with, or after a meal. In some embodiments, the composition is directly introduced into the subject's gastrointestinal tract, e.g., by colonoscopy.

In the second aspect, the present invention provides a method for treating obesity in an obese subject or a subject at risk of obesity by introducing into the gastrointestinal tract of the subject an effective amount of (1) one or more of Schizosaccharomyces pombe, Cryptococcus neoformans, and Saccharomyces cerevisae; or (2) an inhibitor of one or more of Lachancea thermotolerans, Saccharomyces paradoxus, Thielavia terrestris, Parastagonospora nodorum, and Myceliophthora thermophila. In some embodiments, introduced into the subject's gastrointestinal tract is an effective amount of (1) one or more of Schizosaccharomyces pombe and Cryptococcus neoformans; or (2) an inhibitor of one or more of Lachancea thermotolerans, Saccharomyces paradoxus, and Thielavia terrestris. In some embodiments, an effective amount of one or both of Schizosaccharomyces pombe and Cryptococcus neoformans is introduced into the subject's gastrointestinal tract, e.g., by way of colonoscopy.

In some embodiments, the level of Schizosaccharomyces pombe, Cryptococcus neoformans, and/or Saccharomyces cerevisae is increased by about 4-10 fold after the introducing step. In some embodiments, the relative level of Schizosaccharomyces pombe is greater than about 3.99% of total gut fungi in the gastrointestinal tract of the subject after the introducing step. In some embodiments, the relative level of Lachancea thermotolerans, Saccharomyces paradoxus, Thielavia terrestris, Parastagonospora nodorum, and/or Myceliophthora thermophila is reduced to less than about 0.1% of total gut fungi in the gastrointestinal tract of the subject after the introducing step. In some embodiments, the fungal alpha diversity index is increased by about 1.2 to 6 fold after the introducing step.

In some embodiments, the claimed method comprises introducing into the gastrointestinal tract of the subject a composition comprising: A. (i) one or more of Schizosaccharomyces pombe, Cryptococcus neoformans, and Saccharomyces cerevisae; or (ii) an inhibitor of one or more of Lachancea thermotolerans, Saccharomyces paradoxus, Thielavia terrestris, Parastagonospora nodorum, and Myceliophthora thermophila, and B. one or more physiologically acceptable excipients. In some embodiments, the composition comprises one or both of Schizosaccharomyces pombe and Cryptococcus neoformans. In some embodiments, the composition is directly delivered to the subject's small intestine, ileum, or large intestine, e.g., by colonoscopy. In some embodiments, the composition is formulated for oral administration by the subject. In some embodiments, the composition is formulated in a daily dosage to be administered each day. In some embodiments, the composition is in the form of a food or beverage item. In some embodiments, the composition further comprises a prebiotic, such as inulin or psylluim husk. In some embodiments, the introducing step is performed by fecal microbiota transplantation (FMT). In some embodiments, the Schizosaccharomyces pombe, Cryptococcus neoformans, and/or Saccharomyces cerevisae is derived and partially purified from a donor's fecal material or is obtained from culture. In some embodiments, the subject's bodyweight is reduced, serum low-density lipoprotein-cholesterol (LDL-C) level is reduced, serum glucose level is reduced, glucose tolerance is increased, and/or insulin responsiveness is increased.

In a related aspect, the present invention provides a novel use of a composition for treating obesity and associated conditions such as metabolic syndrome, type 2 diabetes mellitus (T2DM), fatty liver, hypertension, cardiovascular disease etc. or for preventing these conditions in a subject. The composition contains an effective amount of (1) one or more of Schizosaccharomyces pombe, Cryptococcus neoformans, and Saccharomyces cerevisae; or an inhibitor of one or more of Lachancea thermotolerans, Saccharomyces paradoxus, Thielavia terrestris, Parastagonospora nodorum, and Myceliophthora thermophila, and (2) one or more physiologically acceptable excipients. In some embodiments, the composition consists essentially of, or consists of, the specified components such as (1) one or more of Schizosaccharomyces pombe, Cryptococcus neoformans, and Saccharomyces cerevisae; or an inhibitor of one or more of Lachancea thermotolerans, Saccharomyces paradoxus, Thielavia terrestris, Parastagonospora nodorum, and Myceliophthora thermophila, and (2) one or more physiologically acceptable excipients. In some embodiments, the composition contains an effective amount of (1) Schizosaccharomyces pombe and/or Cryptococcus neoformans; or (2) an inhibitor of one or more of Lachancea thermotolerans, Saccharomyces paradoxus, and Thielavia terrestris. In some embodiments, the composition comprises an effective amount of Schizosaccharomyces pombe and/or Cryptococcus neoformans plus one or more physiological acceptable excipients. In some embodiments, the composition further includes at least one prebiotic, e.g., inulin and/or psyllium husk. In some embodiments, the composition is in the form of a food item or a beverage, which may be taken before, with, or after a meal. In some embodiments, the composition is directly introduced into the subject's gastrointestinal tract, e.g., by colonoscopy.

In a third aspect, the present invention provides a kit for promoting weight loss or reducing risk of obesity in a subject comprising a plurality of compositions each comprising an effective amount of one or more of the following: Schizosaccharomyces pombe; Cryptococcus neoformans; Saccharomyces cerevisae; an inhibitor of Lachancea thermotolerans; an inhibitor of Saccharomyces paradoxus; an inhibitor of Thielavia terrestris; an inhibitor of Parastagonospora nodorum, and an inhibitor of Myceliophthora thermophila. In some embodiments, the kit includes a first composition, which comprises an effective amount of Schizosaccharomyces pombe and/or Cryptococcus neoformans, and a second composition, which comprises an inhibitor of Lachancea thermotolerans, Saccharomyces paradoxus, and/or Thielavia terrestris. In some embodiments, the kit comprises a first composition, which comprises an effective amount of Schizosaccharomyces pombe, and a second composition, which comprises an effective amount of Cryptococcus neoformans. In some embodiments, the compositions contained in the claimed kit may be formulated for oral administration or for direct delivery to the subject's gastrointestinal tract, e.g., by colonoscopy.

### BRIEF DESCRIPTION OF THE DRAWINGS

FIG. 1. Fecal fungal community structure in Hong Kong and Kunming cohorts. (A) Variations in gut mycobiome composition at genus level in association with obesity across two regions. (B) The effect of size of metadata variables in human gut mycobiome variation. *P<0.05, ***P<0.001. (C-E) Variations in the α-diversity of human gut mycobiome in association with obesity across two regions. The fecal fungal diversity (C) evenness (D) and richness (E) were compared between lean and obese groups across two regions. HK, Hong Kong; KM, Kunming. Statistical significance was determined by t-test and the numbers above the bars represent p-value. For box plots, the vertical lines extend 1.5 times the interquartile range (top and bottom borders of the box) and the median depicted by the horizontal line inside the box.

FIG. 2. Variations in gut mycobiome composition (A) and differences in the relative abundance of major fungal taxa (B) at the species level in association with obesity in Hong Kong and Kunming cohorts. Differences in abundance were detected using Linear discriminant analysis Effect Size (LEfSe) analysis. Only dominant species (relative abundance ≥1.0%) and when analyzed with LefSE analysis with FDR correction having adjusted p values of <0.05 and effect size of >2 are shown. Log transformation of the relative abundance were shown in the boxplot. HK, Hong Kong; KM, Kunming. *P<0.05, **P<0.01. P values were determined by Wilcoxon rank-sum test.

FIG. 3. Putative fungal functional profiles in the fecal samples of lean and obese subjects from Hong Kong and Kunming cohorts. (A) Pie chart of the 12 guilds in the fecal samples. Lean in HK, lean individuals in Hong Kong; Obesity in HK, obese individuals in Hong Kong; Lean in KM, lean individuals in Kunming; Obesity in KM, obese individuals in Kunming. (B-E) Relative abundance of animal endosymbiotic (B), epiphytic (C), plant pathogenic (D), plant saprotrophic (E) fungi in the fecal samples. All data are presented as the mean±SEM. The bars with numbers indicate statistical differences. p values are from t-test. HK, Hong Kong; KM, Kunming.

FIG. 4. Co-occurrence network between fungal taxa in the feces of lean and obese subjects from Hong Kong and Kunming cohorts. (A) Lean group from Hong Kong cohorts; (B) Obese group from Hong Kong cohorts; (C) Lean group from Kunming cohorts; (D) Obese group from Kunming cohorts. Colored nodes represent Operational Taxonomic Units (OTUs) assigned to major genus. Edges between nodes stands for either positive (blue) or negative (red) co-occurrence relationships inferred from OTU abundance profiles using the SparCC method (pseudo p<0.05, correlation values <−0.6 or >0.6). HK, Hong Kong; KM, Kunming.

FIG. 5. Correlations between the α diversity (diversity, evenness and richness) of fecal bacteria and fungi in lean and obese subjects from Hong Kong and Kunming cohorts. (A) Lean group from Hong Kong cohorts; (B) Obese group from Hong Kong cohorts; (C) Lean group from Kunming cohorts; (D) Obese group from Kunming cohorts. Spearman's correlation coefficient was calculated, while statistical significance was determined for all pairwise comparisons. Significant correlations (p<0.05) are displayed with asterisk. *p<0.05, **p<0.01, ***p<0.001. Blue circles indicate positive correlations and red circles indicate inverse correlations. The size and shading indicate the magnitude of the correlation where darker shades denote more intensive correlations than light ones. HK, Hong Kong; KM, Kunming.

FIG. 6. The effects of Schizosaccharomyces pombe on obesity and serological parameters. (A) Schematic diagram, (B) Body weight gain, (C) Adiposity index, (D) Subcutaneous adipose tissue weight, (E) Epididymal adipose tissue weight, (F) Perirenal adipose weight, (G) Serum glucose profile of insulin tolerance test (ITT), (H) Area under curve (AUC) measured during ITT, (I) Serum glucose profile of oral glucose tolerance test (OGTT), (J) AUC measured during OGTT. Data are shown as means±SEM (n=4 or 5). p value was corrected with false discovery rate (FDR). *p<0.05, **p<0.01, ***p<0.001, ns indicates no significant difference. p values are from one-way ANOVA. ND: Normal diet; HF: High fat diet; SE: Group treated with Saccharomyces cerevisiae; SP: Group treated with Schizosaccharomyces pombe; OGTT: oral glucose tolerance test; ITT: insulin tolerance test.

FIG. 7. Variations in the mycobiome composition with respect to region and obesity. (A) β-diversity of fecal fungi from obese and lean subjects from Hong Kong and Kunming. NMDS (non-metric multidimensional scaling) plotting based on Bray-Curtis dissimilarities at the fungal species level. Fecal fungal community structure difference between lean and obese subjects in Hong Kong (B) and Kunming (C).

FIG. 8. Variations in the α-diversity of human gut mycobiome according to medications and type 2 diabetes mellitus (T2DM). The impact of mediation on gut mycobiome richness (A) and diversity (B). The impact of type 2 diabetes mellitus on gut mycobiome richness (C) and diversity (D) (as measured in Chao1 richness and Simpson diversity index, respectively). The bars with numbers indicate statistical differences. Statistical significance between Y and N was determined by Wilcoxon. HK, Hong Kong; KM, Kunming. Y, yes; N, no.

FIG. 9. Negative association between Schizosaccharomyces pombe and BMI. (A) Negative association between Schizosaccharomyces pombe and BMI in Hong Kong and Kunming, respectively. (B) The association between major fungal species and BMI. p values are from t-test. HK, Hong Kong; KM, Kunming.

FIG. 10. The difference of relative abundance of putative fungal functional profiles in the fecal samples between Hong Kong and Kunming cohorts. All data are presented as the mean±SEM. The bars with numbers indicate statistical differences. p values are from t-test.

FIG. 11. Liver mass index and the serum lipids levels of the mice. (A) Liver mass index; (B) High density lipoprotein-cholesterol (HDL-C); (C) Low density lipoprotein-cholesterol (LDL-C); (D) Total cholesterol (TC); (E) Total triglyceride (TG). Data are shown as means±SEM (n=4 or 5). *p<0.05, **p<0.01, ***p<0.001, ns indicates no significant difference. p values are from one-way ANOVA.

### DEFINITIONS

The term “fecal microbiota transplantation (FMT)” or “stool transplant” refers to a medical procedure during which fecal matter containing live fecal microorganisms (bacteria, fungi, and the like) obtained from a healthy individual is transferred into the gastrointestinal tract of a recipient to restore healthy gut microflora that has been disrupted or destroyed by a variety of medical conditions. Typically, the fecal matter from a healthy donor is first processed into an appropriate form for the transplantation, which can be made through direct deposit into the lower gastrointestinal tract such as by colonoscopy, or by nasal intubation, or through oral ingestion of an encapsulated material containing dried and frozen fecal matter.

The term “antifungal agent” refers to any substance that is capable of inhibiting, suppressing, or preventing the growth or proliferation of fungal species, either generally or specifically for certain fungal species, e.g., Lachancea thermotolerans, Saccharomyces paradoxus, Thielavia terrestris, Parastagonospora nodorum, or Myceliophthora thermophila. Known agents with general fungicidal activity include amphotericin B, echinocandin, fluconazole, nystatin, and clotrimazole.

“Percentage relative abundance,” when used in the context of describing the presence of a particular fungal species (e.g., Schizosaccharomyces pombe or Cryptococcus neoformans) in relation to all fungal species present in the same environment, refers to the amount of the specific fungal species relative to the amount of all fungal species as expressed in a percentage form. For example, the percentage relative abundance of fungal species Schizosaccharomyces pombe can be determined by comparing the quantity of S. pombe-specific DNA (e.g., determined by quantitative polymerase chain reaction) in one given sample with the quantity of all fungal DNA (e.g., determined by quantitative PCR and sequencing based on the Internal transcribed spacer 2 or ITS2 sequence) in the same sample.

“Absolute abundance,” when used in the context of describing the presence of a particular fungal species (e.g., Schizosaccharomyces pombe or Cryptococcus neoformans) in the feces, refers to the amount of DNA derived from the fungal species out of the amount of all DNA in a fecal sample. For instance, the absolute abundance of Schizosaccharomyces pombe can be determined by comparing the quantity of S. pombe-specific DNA (e.g., determined by quantitative PCR) in one given sample with the quantity of all fecal DNA in the same sample.

“Total fungal load” of a fecal sample, as used herein, refers to the amount of all fungal DNA out of the amount of all DNA in the fecal sample. For instance, the absolute abundance of fungi can be determined by comparing the quantity of fungal specific DNA (e.g., 18S rDNA determined by quantitative polymerase chain reaction) in one given sample with the quantity of all fecal DNA in the same sample.

The term “fungal alpha diversity index,” as used in reference to fungal species, describes the diversity of fungal communities in a predefined sample or site, e.g., a fecal sample taken from a person's gastrointestinal tract. In this disclosure, “alpha diversity” indicates the complexity of the community within each sample, separately taking into consideration both the variety as well as relative abundance of each species: a higher alpha diversity indicates a sample as containing a more complex community, including a higher number of distinct species (variety) and a higher frequencies of the different species (relative abundance of each species). Definition of alpha diversity is often based on the values given by one or more diversity indices, such as species richness (account of the number of different species present in a sample), species evenness (a measure of the degree to which individuals are split among species, with low values indicating that one or a few species dominate, and high values indicating that relatively equal numbers of individuals belong to each species), and the Shannon index or the Simpson index (which take into account species proportional abundances in a community).

As used herein, the term “obese” is used to describe a non-Asian person who has a body mass index (BMI) of 30 or higher and an Asian person who has a BMI of 25 or higher, whereas the term “overweight” is used to describe a non-Asian person who has a BMI of 25 or higher and an Asian person who has a BMI of 23 or higher.

The term “inhibiting” or “inhibition,” as used herein, refers to any detectable negative effect on a target biological process, such as RNA/protein expression of a target gene, the biological activity of a target protein, cellular signal transduction, cell proliferation, presence/level of an organism especially a micro-organism, any measurable biomarker, bio-parameter, or symptom in a subject, and the like. Typically, an inhibition is reflected in a decrease of at least 10%, 20%, 30%, 40%, 50%, 60%, 70%, 80%, 90% or greater in the target process (e.g., a subject's bodyweight, or the blood glucose/cholesterol level, or any measurable symptom or biomarker in a subject, such as an infection rate among subjects by a pathogenic infectious agent), or any one of the downstream parameters mentioned above, when compared to a control. “Inhibition” further includes a 100% reduction, i.e., a complete elimination, prevention, or abolition of a target biological process or signal. The other relative terms such as “suppressing,” “suppression,” “reducing,” and “reduction” are used in a similar fashion in this disclosure to refer to decreases to different levels (e.g., at least 10%, 20%, 30%, 40%, 50%, 60%, 70%, 80%, 90% or greater decrease compared to a control level) up to complete elimination of a target biological process or signal. On the other hand, terms such as “activate,” “activating,” “activation,” “increase,” “increasing,” “promote,” “promoting,” “enhance,” “enhancing,” or “enhancement” are used in this disclosure to encompass positive changes at different levels (e.g., at least about 5%, 10%, 20%, 30%, 40%, 50%, 60%, 70%, 80%, 90%, 100%, 200%, or greater such as 3, 5, 8, 10, 20-fold increase compared to a control level in a target process, signal, or parameter. As used herein, the term “treatment” or “treating” includes both therapeutic and preventative measures taken to address the presence of a disease or condition or the risk of developing such disease or condition at a later time. It encompasses therapeutic or preventive measures for alleviating ongoing symptoms, inhibiting or slowing disease progression, delaying of onset of symptoms, or eliminating or reducing side-effects caused by such disease or condition. A preventive measure in this context and its variations do not require 100% elimination of the occurrence of an event; rather, they refer to a suppression or reduction in the likelihood or severity of such occurrence or a delay in such occurrence.

A “patient” or “subject” receiving the composition or treatment method of this invention is a human, including both adult and juvenile human, of any age, gender, and ethnic background, who may not have been diagnosed with any particular disease or disorder (e.g., have not had a blood glucose test result for a diagnosis of diabetes) but is in need of improving metabolic health (e.g., to reduce one's bodyweight or eliminate one's risk for developing obesity or diabetes). Typically, the patient or subject receiving treatment according to the method of this invention to improve metabolic health, such as weight loss or reducing risk of becoming overweight or obese, is not otherwise in need of treatment by the same therapeutic agents. For example, if a subject is receiving the composition according to the claimed method, the subject is not suffering from any disease that is known to be treated by the same therapeutically active agents. Although a patient may be of any gender and any age, in some cases the patient is at least 40, 45, 50, 55, 60, 65, 70, 75, 80, or 85 years of age; in some cases, a patient may be between 40 and 45 years old, or between 50 and 65 years of age, or between 65 and 85 years of age. A “child” subject is one under the age of 18 years, e.g., about 5-17, 9 or 10-17, or 12-17 years old, including an “infant,” who is younger than about 12 months old, e.g., younger than about 10, 8, 6, 4, or 2 months old, whereas an “adult” subject is one who is 18 years or older.

The term “effective amount,” as used herein, refers to an amount that produces intended (e.g., therapeutic or prophylactic) effects for which a substance is administered. The effects include the prevention, correction, or inhibition of progression of the symptoms of a particular disease/condition and related complications to any detectable extent, e.g., incidence of obesity, type 2 diabetes, hypertension, heart disease, etc., one or more of the symptoms of these conditions and related disorders. The exact “effective” amount for any therapeutic agent will depend on the nature of the agent as well as the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)).

As used herein, the term “about” denotes a range of value that is +/−10% of a specified value. For instance, “about 10” denotes the value range of 10+/−10×10%, i.e., 9 to 11.

A “pharmaceutically acceptable” or “pharmacologically acceptable” excipient is a substance that is not biologically harmful or otherwise undesirable, i.e., the excipient may be administered to an individual along with a bioactive agent without causing any undesirable biological effects. Neither would the excipient interact in a deleterious manner with any of the components of the composition in which it is contained.

The term “excipient” refers to any essentially accessory substance that may be present in the finished dosage form of the composition of this invention. For example, the term “excipients” mat include, but are not limited to, vehicles, buffers, binders, disintegrants, fillers (diluents), lubricants, glidants (flow enhancers), compression aids, colors, sweeteners, preservatives, suspending/dispersing agents, film formers/coatings, flavors and printing inks.

The term “consisting essentially of,” when used in the context of describing a composition containing an active ingredient or multiple active ingredients, refer to the fact that the composition does not contain other ingredients possessing any similar or relevant biological activity of the active ingredient(s) or capable of enhancing or suppressing the activity, whereas one or more inactive ingredients such as physiological or pharmaceutically acceptable excipients may be present in the composition. For example, a composition consisting essentially of active agents (for instance, a composition comprising S. pombe or C. neoformans) effective for bodyweight reduction or improving metabolic health in a subject is a composition that does not contain any other agents that may have any detectable positive or negative effect on the same target process (e.g., weight loss, reduction of blood glucose, lipid, or cholesterol level, improvement on glucose tolerance, or improvement of insulin sensitivity) or that may increase or decrease to any measurable extent of the disease or symptoms among the receiving subjects.

### DETAILED DESCRIPTION OF THE INVENTION

### I. Introduction

The purpose of this invention relates to novel methods and compositions useful for preventing, ameliorating, and treating obesity and obesity related condition that involves specific fungal species. In the present invention, the inventors discovered certain fungal species are at an altered level in subjects with obesity. Health benefits such as prevention and alleviation of obesity and obesity-related conditions can be achieved by modulating the level of these fungal species in patients' gut, for example, by oral administration of the beneficial species or agents capable of suppression of undesirable species. Fecal microbiota transplantation (FMT) technique is one preferred method for practicing the present invention.

### II. Pharmaceutical Compositions and Administration

The present invention provides pharmaceutical compositions comprising an effective amount of one or more of Schizosaccharomyces pombe, Cryptococcus neoformans, and Saccharomyces cerevisae for reducing bodyweight in a person to reduce the risk of obesity or a condition related to obesity such as diabetes, hypertension, or cardiovascular disease. Pharmaceutical compositions of the invention are suitable for use in a variety of drug delivery systems. Suitable formulations for use in the present invention are found in Remington's Pharmaceutical Sciences, Mack Publishing Company, Philadelphia, PA, 17th ed. (1985). For a brief review of methods for drug delivery, see, Langer, Science 249:1527-1533 (1990).

In another aspect of this invention, alternative compositions useful for improving the metabolic health of a person, e.g., for weight loss or for reducing one's risk of developing obesity, contain an inhibitor or a specific anti-fungal agent capable of suppressing the proliferation of at least one, possibly two or more (e.g., three, four, or five) of the following fungal species: Lachancea thermotolerans, Saccharomyces paradoxus, Thielavia terrestris, Parastagonospora nodorum, and Myceliophthora thermophila. Such inhibitor or anti-fungal agent preferably is not a broad-spectrum fungicide; rather, it should be a specific anti-fungal agent specifically targeting one or more of the above named species. For example, it may be short polynucleotide in nature of, e.g., a small inhibitory RNA, microRNA, miniRNA, lncRNA, or an antisense oligonucleotide, that is capable of disrupting the expression of at least one key gene in the life cycle of the target fungal species, such that the agent is capable of specifically targeting the species only without significantly affecting other closely related fungal species.

The pharmaceutical compositions of the present invention can be administered by various routes, e.g., systemic administration via oral ingestion or local delivery using a rectal suppository. For example, the preferred route of administering the pharmaceutical compositions is oral administration at daily doses of about 10⁶ to about 10¹² CFU for one or more of Schizosaccharomyces pombe, Cryptococcus neoformans, and Saccharomyces cerevisae. Optionally, the compositions further contain at least one prebiotic such as inulin or psyllium. Additionally, the compositions may be formulated in a daily dosage format. The appropriate dose may be administered in a single daily dose or as divided doses presented at appropriate intervals, for example as two, three, four, or more subdoses per day. The duration of administration may range from about 2 weeks to about 4 weeks, e.g., about 1 week to about 2 weeks.

For preparing pharmaceutical compositions of this invention, one or more inert and pharmaceutically acceptable carriers are used. The pharmaceutical carrier can be either solid or liquid. Solid form preparations include, for example, powders, tablets, dispersible granules, capsules, cachets, and suppositories. A solid carrier can be one or more substances that can also act as diluents, flavoring agents, solubilizers, lubricants, suspending agents, binders, or tablet disintegrating agents; it can also be an encapsulating material.

In powders, the carrier is generally a finely divided solid that is in a mixture with the finely divided active component, e.g., one or more of Schizosaccharomyces pombe, Cryptococcus neoformans, and Saccharomyces cerevisae. In tablets, the active ingredient is mixed with the carrier having the necessary binding properties in suitable proportions and compacted in the shape and size desired.

For preparing pharmaceutical compositions in the form of suppositories, a low-melting wax such as a mixture of fatty acid glycerides and cocoa butter is first melted and the active ingredient is dispersed therein by, for example, stirring. The molten homogeneous mixture is then poured into convenient-sized molds and allowed to cool and solidify.

Powders and tablets preferably contain between about 5% to about 100% by weight of the active ingredient(s) (e.g., one or more of Schizosaccharomyces pombe, Cryptococcus neoformans, and Saccharomyces cerevisae). Suitable carriers include, for example, magnesium carbonate, magnesium stearate, talc, lactose, sugar, pectin, dextrin, starch, tragacanth, methyl cellulose, sodium carboxymethyl cellulose, a low-melting wax, cocoa butter, and the like.

The pharmaceutical compositions can include the formulation of the active ingredient(s) e.g., one or more of Schizosaccharomyces pombe, Cryptococcus neoformans, and Saccharomyces cerevisae, with encapsulating material as a carrier providing a capsule in which the active ingredient(s) (with or without other carriers) is surrounded by the carrier, such that the carrier is thus in association with the active ingredient(s). In a similar manner, sachets can also be included. Tablets, powders, sachets, and capsules can be used as solid dosage forms suitable for oral administration.

Liquid pharmaceutical compositions include, for example, solutions suitable for oral administration or local delivery, suspensions, and emulsions suitable for oral administration. Water-based solutions made from adding into previously sterilized aqueous solutions the active component(s) (e.g., one or more of Schizosaccharomyces pombe, Cryptococcus neoformans, and Saccharomyces cerevisae) in solvents comprising water, buffered water, saline, PBS, ethanol, or propylene glycol are examples of liquid or semi-liquid compositions suitable for oral administration or local delivery such as by rectal suppository. The compositions may contain one or more pharmaceutically acceptable auxiliary substances as required to approximate physiological conditions, such as pH adjusting and buffering agents, tonicity adjusting agents, wetting agents, detergents, and the like.

Sterile solutions (e.g., aqueous solutions) can be first prepared and then the active component(s) (e.g., one or more of Schizosaccharomyces pombe, Cryptococcus neoformans, and Saccharomyces cerevisae, which may be obtained from a culture or may have been inactivated, for example, by heat or by chemical treatment) are added into the desired solvent system. Depending on the nature of the active ingredients/components, different methods can be used to prepare a suitable solvent-based delivery system. For example, one can first dissolve an active compound or compounds in a solution and then pass the resulting solution through a membrane filter to sterilize it or, alternatively, one can dissolve the previously sterilized active compound or compounds in a previously sterilized solvent under sterile conditions. In the case of active components being one or more microorganisms, they can be optionally first purified to remove irrelevant microorganism species to a substantially undetectable level (no greater than 110%, 120% or 150% of background signal, for example) and then placed in a sterile solution to produce a solution-based delivery system. The resulting aqueous solutions may be packaged for use as is, or lyophilized, the lyophilized preparation being combined with a sterile aqueous carrier prior to administration. The pH of the preparations typically will be between 3 and 11, more preferably from 5 to 9, and most preferably from 7 to 8.

Single or multiple administrations of the compositions can be carried out with dose levels and pattern being selected by the treating physician. In any event, the pharmaceutical formulations should provide a quantity of the active agent(s) sufficient to effectively enhance the efficacy of prophylactic or therapeutic regime, such as a program of diet, physical exercise, and other life-style changes, and/or to reduce or eliminate undesirable effects from a weight-related condition (such as type 2 diabetes, hypertension, heart disease, etc.).

### III. Kits

The invention also provides kits for improving the metabolic health of an individual or for preventing a disease associated with obesity according to the method disclosed herein. The kits typically include a plurality of containers, each containing a composition comprising (1) one or more of Schizosaccharomyces pombe, Cryptococcus neoformans, and Saccharomyces cerevisae; (2) an inhibitor of one or more of Lachancea thermotolerans, Saccharomyces paradoxus, Thielavia terrestris, Parastagonospora nodorum, and Myceliophthora thermophila. Optionally, additional ingredient(s) may be included in the composition(s) such as one or more of prebiotics (e.g., inulin or psyllium husk). Further, additional agents or drugs that are known to be therapeutically effective for prevention and/or treatment of pertinent conditions, especially metabolic diseases associated with obesity, including for ameliorating the symptoms and reducing the severity of such diseases, as well as for facilitating recovery from the diseases (such as those described in the last section or otherwise known in the pertinent technical field) may be included in the kit. The plurality of containers of the kit each may contain a different active agent/drug or a distinct combination of two or more of the active agents or drugs.

The kit may further include informational material providing instructions on how to dispense the pharmaceutical composition(s), including description of the type of patients who may be treated (e.g., human patients who are overweight or obese or have been deemed as with high risk of developing a metabolic disease associated with overweightness or obesity and are therefore seeking to improve metabolic health, as well as the subpopulation of patients not to be included in the claimed method, e.g., those who have been diagnosed with a pre-existing condition that already requires the administration of the active components such as one or more of Schizosaccharomyces pombe, Cryptococcus neoformans, and Saccharomyces cerevisae, or an inhibitor of one or more of Lachancea thermotolerans, Saccharomyces paradoxus, Thielavia terrestris, Parastagonospora nodorum, and Myceliophthora thermophila), the dosage, frequency, and manner of administration, and the like.

### EXAMPLES

The following examples are provided by way of illustration only and not by way of limitation. Those of skill in the art will readily recognize a variety of non-critical parameters that could be changed or modified to yield essentially the same or similar results.

### Methods

### Study Subjects

Obese subjects aged 18-70 years with a body mass index (BMI) of ≥28 kg/m² and lean subjects aged 18-70 years with BMI between 18.5 and 23 kg/m² were recruited from Hong Kong and Kunming. Subjects were excluded if they had current severe gastrointestinal diseases, including inflammatory bowel diseases (IBD), cancer, advanced adenoma, or gastroenteritis; or had frequently symptoms of digestive system, including hemafecia, diarrhea, abdominal pain, or vomit; or had current rheumatic immune diseases, active infection, or acquired immunodeficiency syndrome. Subjects were excluded if they had a known history of organ dysfunction or failure including cardiopulmonary system, or urinary system, etc.; or had a known history of abdominal surgery, radio-chemotherapy, or immunotherapy; or had current incurable cancer. Subjects who were pregnant or planning a pregnancy within 1 year were also excluded.

Written informed consent were obtained from all subjects. Their information about age, gender, BMI, alcohol intake, smoking, type 2 diabetes mellitus (T2DM), and medication were recorded. Fecal samples from the study subjects were collected and then stored at −80° C. for downstream gut mycobiome analysis. This study was approved by The Joint Chinese University of Hong Kong-New Territories East Cluster Clinical Research Ethics Committee (No: 2014.026) and The Clinical Research Ethics Committee of Kunming Medical University (No: 2019L23).

### DNA Extraction, Library Preparation and Metagenomic Sequencing

DNA extraction was performed according to methods reported by Zuo, et al.¹ with minor modification. 100 mg of fecal sample was prewashed with 1 ml ddH₂O and pelleted by centrifugation at 13,000×g for 1 min. The fecal pellet was resuspended in 800 μL TE buffer (pH 7.5), supplemented with 16 μL β-mercaptoethanol and 250 U lyticase (Sigma), and incubated at 37° C. for 90 min. The sample was then centrifuged at 13,000×g for 3 min and fecal DNA was subsequently extracted from the pellet using Maxwell®RSC PureFood GMO and Authentication Kit (Promega, Madison, Wisconsin) following manufacturer's instructions. Briefly, 800 mL of cetrimonium bromide (CTAB) buffer was added to the fecal pellet and vortexed for 30 seconds, then the mixture was incubated at 95° C. for 5 minutes. After that, the samples were vortexed thoroughly with beads for 15 min. Next, 40 μL of proteinase K and 20 μl of RNase A were added into sample and the mixture was incubated at 70° C. for 10 minutes. The supernatant was then obtained by centrifuging at 13,000×g for 5 min and was added in Maxwell® RSC machine for DNA extraction. DNA libraries were constructed through the processes of end repairing, purification, and PCR amplification. Whole-genome shotgun sequencing of samples were carried out on Illumina NovaSeq 6000 (Illumina, San Diego, California, USA) with paired-end 150 bp sequencing strategy by Novogene, Beijing, China.

### Bioinformatics and Statistical Analyses

Adaptor contamination and low-quality reads were discarded from the raw data with Kneaddata (website: huttenhower.sph.harvard.edu/kneaddata). Fungal sequence annotation was conducted via HumanMycobiomeScan² and bacteria taxonomic profiling was performed using MetaPhlAn2 classifier³. FUNGuild (website: github. com/UMNFuN/FUNGuild) database was used to annotate fungal functions⁴. The mycobiome and bacteriome abundance table were imported into R (Version 3.6.1) for statistical analysis.

α-diversity indices including observed species, chao1, Simpson and Shannon diversity index were calculated and visualized with R package Vegan 2.5-6. Non-metric multi-dimensional scaling (NMDS) was performed also using the Vegan package. Region, BMI, gender, age, lifestyle (alcohol intake and smoking), type 2 diabetes mellitus (T2DM) factors were examined to explore which one was associated with the establishment of the mycobiome by permutational multivariate analysis of variance (PERMANOVA) using the R package Vegan 2.5-6. Linear discriminant analysis effect size (LEfSe) was used to identify the biomarkers associated with obesity and leanness in both Hong Kong and Kunming. Those with a linear discriminant analysis (LDA) score ≥2.0 were considered to be important biomarkers associated with obesity and leanness.

To identify positive and negative correlations between individual operational taxonomic units (OTUs), sparse correlations for compositional data (SparCC) was developed for quantifying intra- and inter-kingdom relationships. The co-occurrence and co-exclusion networks within fungi were then visualized using Cytoscape 3.7.2. Intra- and inter-kingdom correlations matrix analysis were created using the R package Corrplot.

Student's t test and Wilcoxon rank sum test were used where appropriate to determine the statistically significant differences between different groups, and adjusted with Bonferroni correction for multiple comparisons

### Animal Experiments

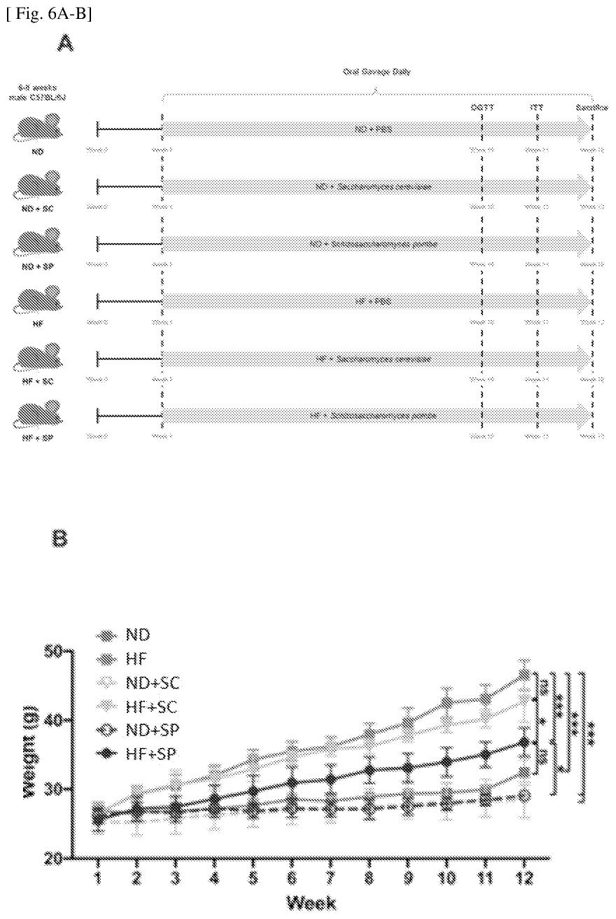

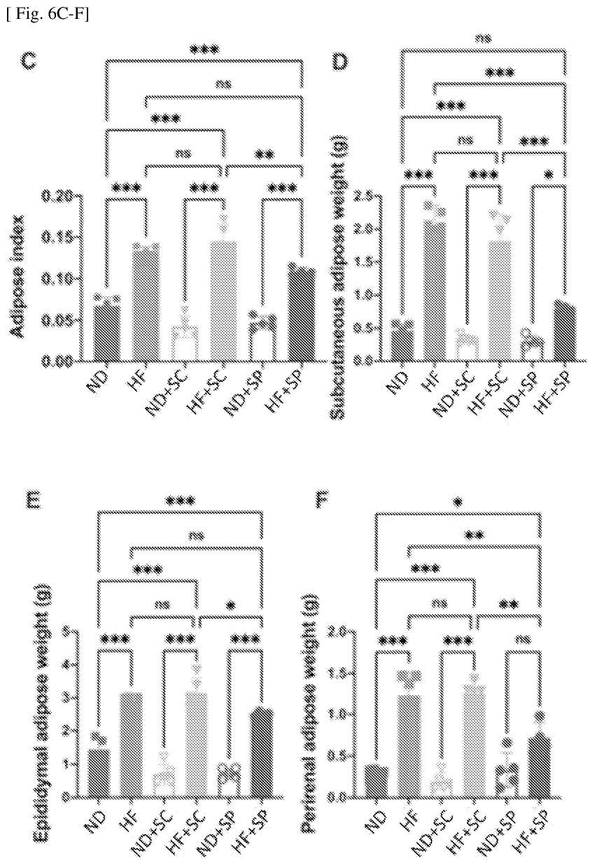

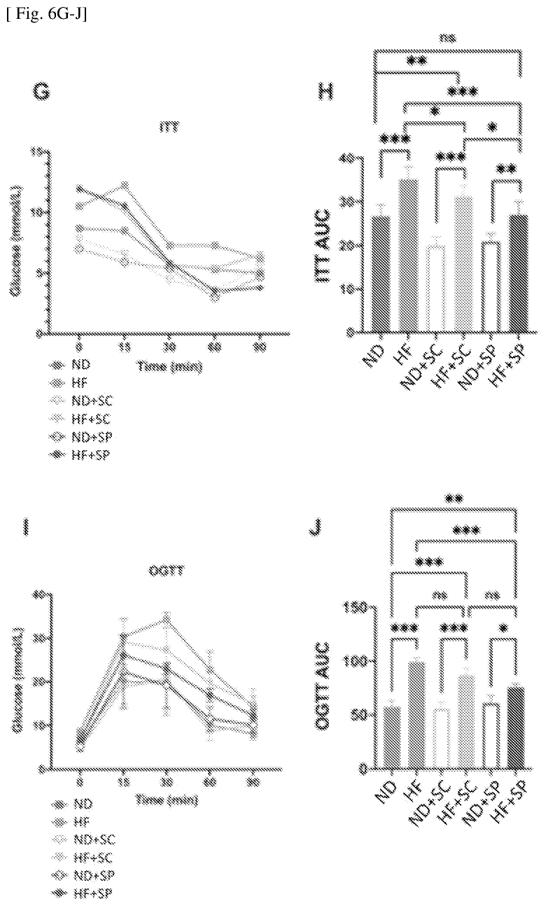

Twenty-nine male C57BL/6J mice (6-8-week-old, n=4 or 5 per group) were obtained from the Laboratory Animal Services Centre at The Chinese University of Hong Kong. All animals were housed in standard plastic cages (four or five mice per cage) under a 12 h light-dark cycle at constant temperature and humidity (23±1° C. and 55±5%, respectively) with free access to food and water. The schematic is shown in FIG. 6A. After acclimatization for 1 week, the mice were divided into six groups (n=4 or 5 per group), three groups received a normal diet (ND) (Labdiet Picolab Select Mouse diet 50 IF/9F Irradiated, containing 18% protein, 8.0% fat and 5% fiber by weight; P. O. BOX 19788, BRENTWOOD, MO 63144), and simultaneously were administrated with 0.2 mL of phosphate-buffered saline (PBS) (ND), 0.2 mL of PBS containing Saccharomyces cerevisiae (ATCC 36375, 1×10⁷ CFU/mouse) (ND+SC), or 0.2 mL of PBS containing Schizosaccharomyces pombe (ATCC 16979, 1×10⁷ CFU/mouse) (ND+SP) respectively for 11 weeks by gavage. Another three groups received a high-sucrose cholesterol diet (HF) (SF11-078, 23% fat, 42.4% sucrose and 19.4% protein by weight; Specialty Feeds, Perth, Australia) and simultaneously were administrated with 0.2 mL of phosphate-buffered saline (PBS) (HF), 0.2 mL of PBS containing Saccharomyces cerevisiae (ATCC 36375, 1×10⁷ CFU/mouse) (HF+SC), or 0.2 mL of PBS containing Schizosaccharomyces pombe (ATCC 16979, 1×10⁷ CFU/mouse) (HF+SP) respectively for 11 weeks by gavage. Body weight of each individual mice was measured weekly.

At week 10 (FIG. 6A), glucose intolerance was evaluated using the standard oral glucose tolerance test (OGTT). Briefly, after 14 h of fasting, a 20% (w/v) glucose solution was administered to mice at a dose of 1 g/kg body weight by oral gavage. Blood samples were collected from a tail cut (by removing the distal 2 mm of the tail) before (0) and at 15, 30, 60 and 90 min after glucose administration. Glucose levels were analyzed with a handheld glucometer (Sannuo; Changsha Sinocare, Inc., Changsha, China). One week later, an insulin tolerance test (ITT) was performed. Diluted regular human insulin (Sigma-Aldrich) was injected intraperitoneally into mice at a dose of 0.75 units/kg body weight under non-fasting conditions. Blood samples were collected from a tail cut (by removing the distal 2 mm of the tail) before (0) and 15, 30, 60 and 90 min after insulin injection, the glucose levels were also analyzed with the handheld glucometer. At the end of the 12-week experiment (FIG. 6A), mice were euthanized by cervical dislocation. Liver and adipose tissues (subcutaneous, epididymal and perirenal) were dissected precisely and weighed. The liver index was defined as the ratio of liver weight divided by the body weight. The adipose index was defined as the ratio of adipose weight divided by the body weight. Total cholesterol (TC), total triglyceride (TG), high-density lipoprotein cholesterol (HDL-C) and low-density lipoprotein cholesterol (LDL-C) levels in the serum were measured using commercial enzymatic kits purchased from Nanjing Jiancheng Bioengineering Institute (Nanjing, China) according to the manufacturer's instructions. All animal experiments were approved and performed in compliance with the Animal Experimentation Ethics Committee (AEEC) of The Chinese University of Hong Kong.

S. pombe and S. cerevisiae Culture

S. pombe were grown in 37° C., 200 rpm in the yeast and mould agar medium (3 g/L yeast extract, 3 g/L malt extract, 5 g/L peptone, 10 g/L dextrose, 20 g/L agar for the solid medium, pH 6.2±0.2). Subsequently, the cultures were frozen and stored with glycerol at −80° C. On the day before administration by oral gavage, the glycerol stocks were thawed and cultivated overnight under the same condition, which was then diluted to the end concentration on the day of oral gavage.

Similarly, S. cerevisiae were grown in 37° C., 200 rpm in the yeast peptone dextrose (YPD) medium (20 g/L glucose, 20 g/L peptone, 10 g/L yeast extract, 20 g/L agar for the solid medium). Subsequently, the cultures were frozen and stored with glycerol at −80° C. On the day before administration by oral gavage, the glycerol stocks were thawed and cultivated overnight under the same condition, which was then diluted to the end concentration on the day of oral gavage.

### Results

### Clinical Characteristics of Study Subjects

A total of 217 subjects from two regions (Hong Kong and Kunming) in China were included in this study. 118 subjects (59 lean and 59 obese subjects) were recruited in Hong Kong and 99 subjects (49 lean and 50 obese subjects) were recruited in Kunming. In the Hong Kong cohort, the median age of lean and obese participants was 45.88 and 47.85 years, respectively. In the Kunming cohort, the median age was 48.14 and 40.56 years for lean and obese participants, respectively. Smoking status, T2DM and medication use were significantly different between lean and obese subjects in Hong Kong cohort, and the age and gender were significantly different between lean and obese subjects in Kunming cohort. Other clinical characteristics are comparable (Table 1).

TABLE 1
Clinical Characteristics of Study Subjects

| Cohort | Factor | Lean | Obesity | p values ^{a} |
|---|---|---|---|---|
| Hong Kong | Sample size, n | 59 | 59 | NA |
| (n = 118) | BMI ^{b, c}, kg/m2 | 20.99 ± | 35.91 ± | <0.001 |
| | | 1.61 | 5.47 | |
| | Age ^{c}, year | 45.88 ± | 47.85 ± | 0.5780 |
| | | 13.69 | 11.51 | |
| | Male, n | 32 | 37 | 0.4551 |
| | Alcohol, n | 8 | 5 | 0.6536 |
| | Smoking, n | 6 | 4 | 0.0276 |
| | T2DM, n | 2 | 42 | <0.0001 |
| | Medication use^{d}, n | 2 | 51 | <0.0001 |
| Kunming | Sample size, n | 49 | 50 | NA |
| (n = 99) | BMI ^{b, c} | 20.69 ± | 31.09 ± | <0.0001 |
| | | 1.18 | 3.51 | |
| | Age ^{c} | 48.14 ± | 40.56 ± | 0.0027 |
| | | 12.94 | 13.66 | |
| | Male, n | 15 | 33 | 0.0177 |
| | Alcohol, n | 6 | 13 | 0.1287 |
| | Smoking, n | 10 | 9 | 0.5937 |
| | T2DM, n | 0 | 6 | 0.0853 |
| | Medication use^{d}, n | 21 | 21 | >0.9999 |

^{a} From Wilcoxon rank-sum test for continuous variables and Fisher's exact test for categorical variables;
^{b} BMI, Body Mass Index.
^{c} Expressed in mean ± SD or median (25th to 75th percentile, P25-P75) as appropriate
^{d}Any medications including metformin, sulfonylureas, statins, proton-pump inhibitors, and nonsteroidal anti-inflammatory, antibiotics, angiotensin-converting-enzyme inhibitors, and calcium channel blocker.

### Variation of Gut Mycobiome Composition in Lean and Obese Individuals Across Regions

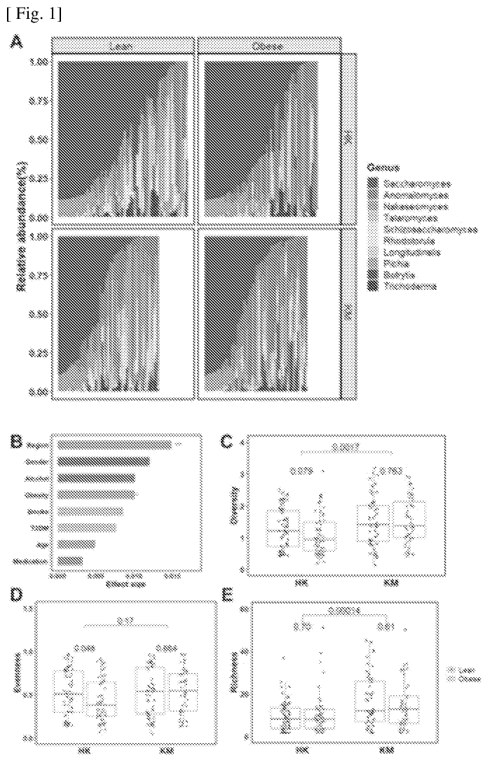

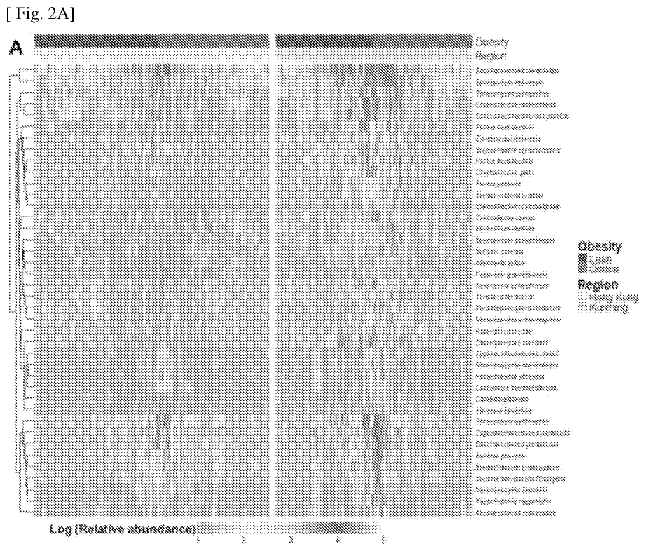

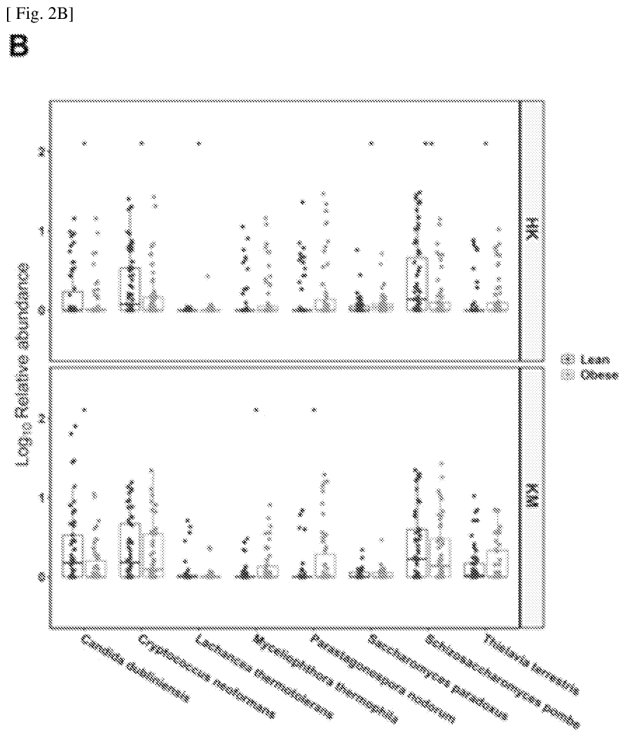

At the phylum level, Ascomycota was the most predominant fungi detected in all samples, while at the genus level, Saccharomyces was the most prevalent fungi detected in both the Hong Kong (mean relative abundance: 48.53%) and Kunming (mean relative abundance: 34.85%) individuals (FIG. 1A). The most abundant species were Saccharomyces cerevisiae (detected in 90.68% of Hong Kong subjects, and 86.87% of Kunming subjects) and Sporisorium reilianum (detected in 84.75% of Hong Kong subjects, and 95.96% of Kunming subjects) (FIG. 2A). Prevalence of other fungal species are shown in Table 2.

TABLE 2
Prevalence of selected gut fungal species
detected in Hong Kong and Kunming cohorts

| Species | Hong Kong | Kunming |
|---|---|---|
| Candida dubliniensis | 36.44% | 55.56% |
| Cryptococcus neoformans | 54.24% | 67.68% |
| Lachancea thermotolerans | 18.64% | 30.30% |
| Myceliophthora thermophila | 23.73% | 37.37% |
| Parastagonospora nodorum | 29.66% | 32.32% |
| Schizosaccharomyces pombe | 52.54% | 68.69% |
| Thielavia terrestris | 30.51% | 51.52% |

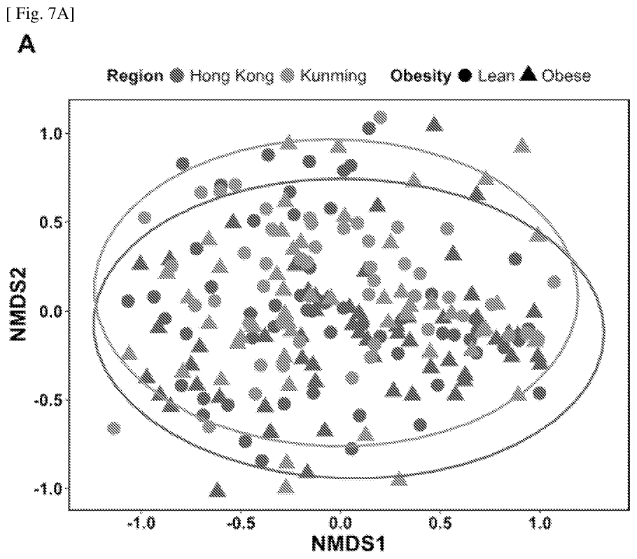

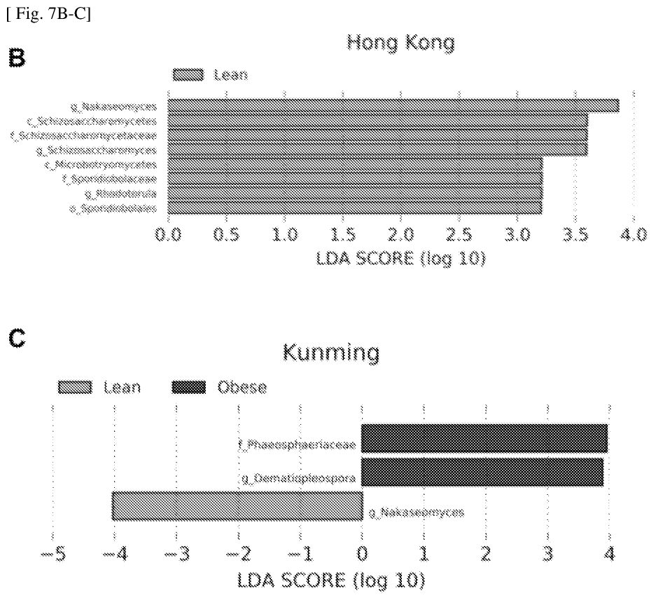

Amongst all clinical variables (region, gender, alcohol intake, obesity, smoke, T2DM, age and medication), region (effect size R²=0.015, p<0.001) and obesity (effect size R² =0.010, p<0.05) showed the largest effect sizes on fecal mycobiome variation (FIG. 1B, Table 3). By non-metric multi-dimensional scaling (NMDS) analysis, Kunming subjects (yellow dots) cluster separate of Hong Kong subjects (blue dots), reinforcing the finding of significant mycobiome compositional difference between the Hong Kong and Kunming subjects (PERMANOVA test, p<0.001, FIG. 7A).

TABLE 3
Effect of obesity, region, gender, diabetes, smoking and
alcohol on the gut mycobiota analyzed by PERMANOVA

| Factor | d.f. | R² | F | P value |
|---|---|---|---|---|
| Region | 1 | 0.014846 | 3.239889 | <0.001*** |
| Gender | 2 | 0.011992 | 1.298706 | 0.251 |
| Alcohol | 3 | 0.01006 | 0.70117 | 0.788 |
| Obesity | 1 | 0.01005 | 2.1215 | 0.046* |
| Smoke | 2 | 0.00853 | 0.89435 | 0.502 |
| T2DM | 1 | 0.00763 | 1.6068 | 0.055 |
| Age | 1 | 0.00486 | 1.0197 | 0.35 |
| Medication | 1 | 0.00322 | 0.6787 | 0.677 |

Fecal mycobiome diversity between regions and between obese versus lean subjects was then evaluated. Fecal mycobiome diversity and richness were significantly higher in the Kunming subjects than that in the Hong Kong subjects (t-test, p<0.01 and p<0.001, respectively, FIGS. 1C and E). Regarding obesity, a lower fecal fungal evenness but not diversity was observed in obese subjects compared to lean subjects in the Hong Kong cohort (t-test, p<0.05 and p=0.079, respectively, FIGS. 1C and D). A more homogeneous fecal fungal composition was shown, however, between lean and obese individuals in Kunming cohorts (t-test, p=0.763 in diversity; p=0.664 in evenness; p=0.61 in richness, respectively, FIG. 1C-E). Collectively, the disrupted pattern of gut mycobiota in obesity subjects varied across regions compared with that of lean individuals.

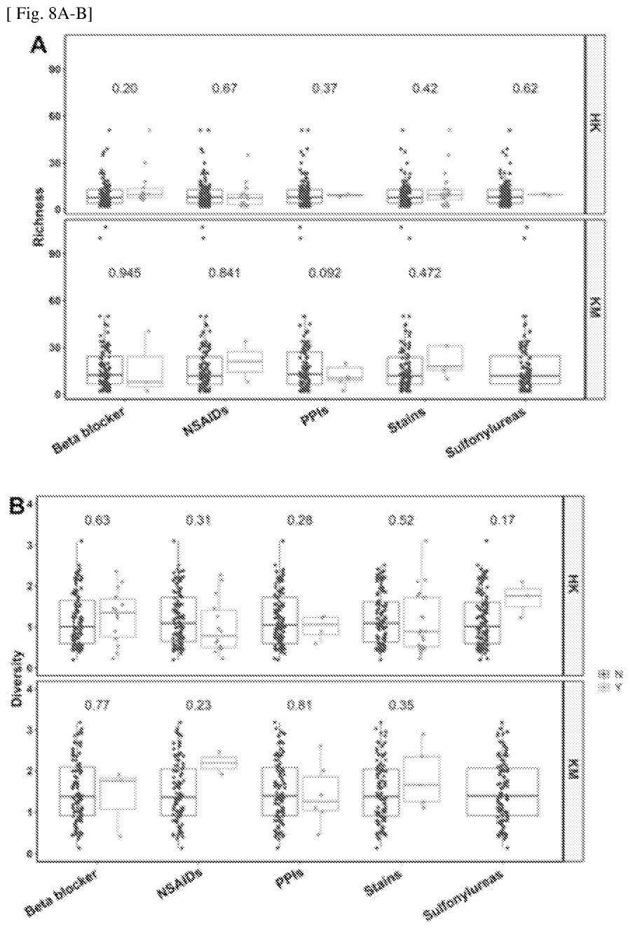

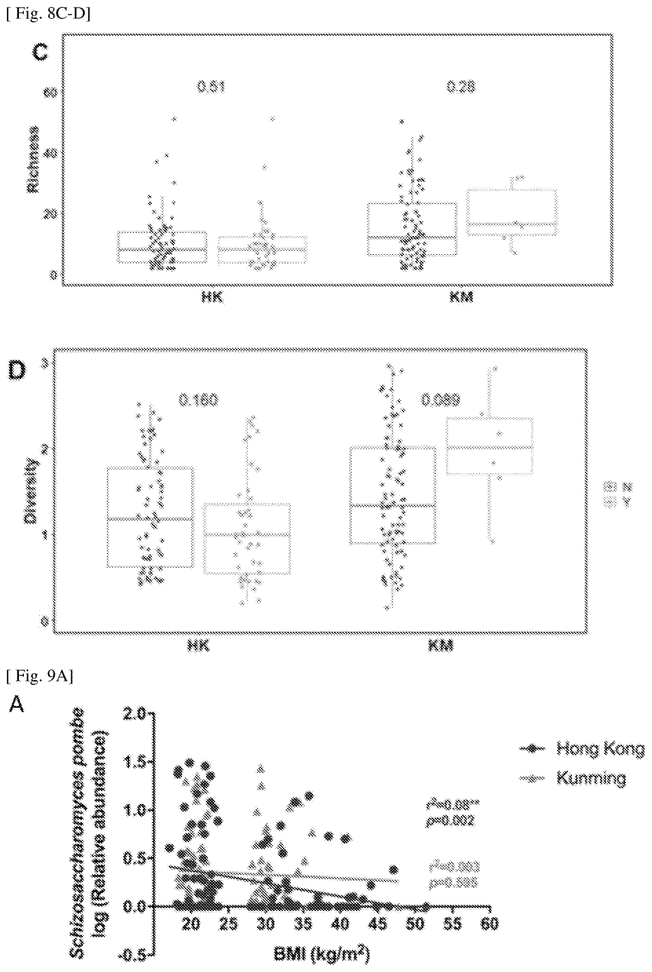

In addition, the associations between medications and gut mycobiome composition was further explored. Beta blocker, non-steroidal anti-inflammatory drugs (NSAIDs), proton-pump inhibitors (PPIs), statins and sulfonylureas (SUs) were recorded in the medical history of participants included (FIG. 8). The results showed that there was no significant correlation between use of these five medications and fungal diversity or richness (FIGS. 8A and B). Moreover, T2DM did not show significant impact on fecal fungal composition (FIGS. 8C and D).

### Fecal Fungal Trophic Modes and Functional Groups Prediction in Lean and Obese Individuals Across Regions

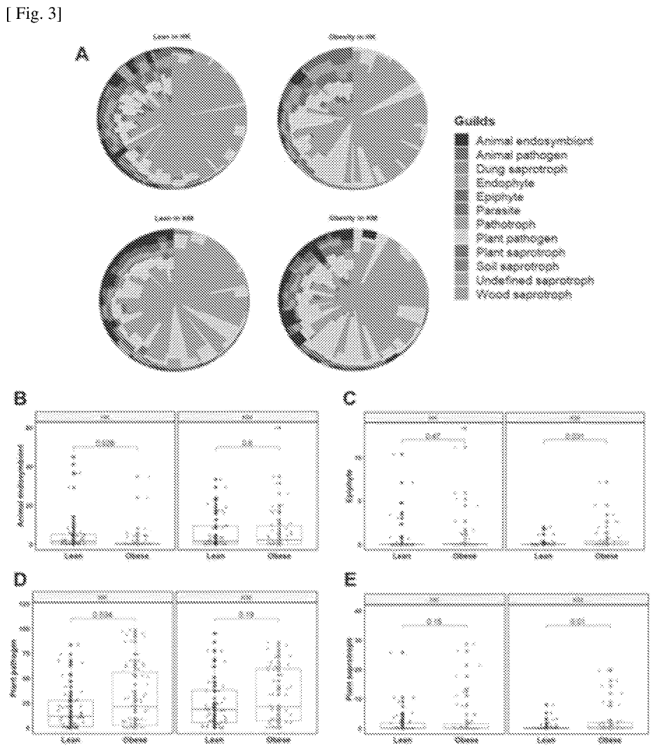

To further explore how obesity and region affect the function of the gut mycobiome, fecal mycobiome trophic modes and ecological guild prediction were performed on obese and lean subjects in relation to region (FIG. 3). According to classification based on nutrient substrate preferences, the fungi were divided into twelve guilds (dung saprotroph, wood saprotroph, soil saprotroph, animal pathogen, plant pathogen, epiphyte, endophyte, animal endosymbiont, fungal parasite and undefined saprotroph) and three trophic modes (pathotroph, saprotroph and symbiotroph) (FIG. 3A). Most of the detected fungi in the fecal mycobiome were saprotrophic, contributing over 79% of the community, predominantly including of taxon Saccharomycesles.

Fungal function varied largely across geographic regions (FIG. 3). Animal endosymbiotic (t-test, p<0.05), animal pathogenic (t-test, p<0.05), endophytic (t-test, p<0.01), parasitic (t-test, p<0.05), soil saprotrophic (t-test, p<0.05) and wood saprotrophic (t-test, p<0.01) fungi were more abundant in fecal mycobiomes of Hong Kong subjects compared with that of Kunming subjects (FIG. 10). The trophic mode of fecal mycobiome also showed significant association with obesity which was also region-specific (FIG. 3). In Hong Kong, the contents of animal endosymbiotic fungi were much higher (t-test, p<0.05) while plant pathogenic fungi were less (t-test, p<0.05) in the feces of lean cohorts (FIGS. 3B and D). In Kunming, epiphytic and plant saprotrophic fungi were relatively more abundant in the feces of obese group than in the lean group, displaying a higher relative abundance of about 74.48% (Wilcoxon rank-sum test, p<0.05) and 79.24% (Wilcoxon rank-sum test, p=0.01) respectively (FIGS. 3C and E). These data imply that region and obesity have a combined impact on the gut fungal functionality landscape.

### Decreased Fungal-Fungal Intra-Kingdom Ecological Interactions in the Fecal Mycobiome in Obesity Across Regions

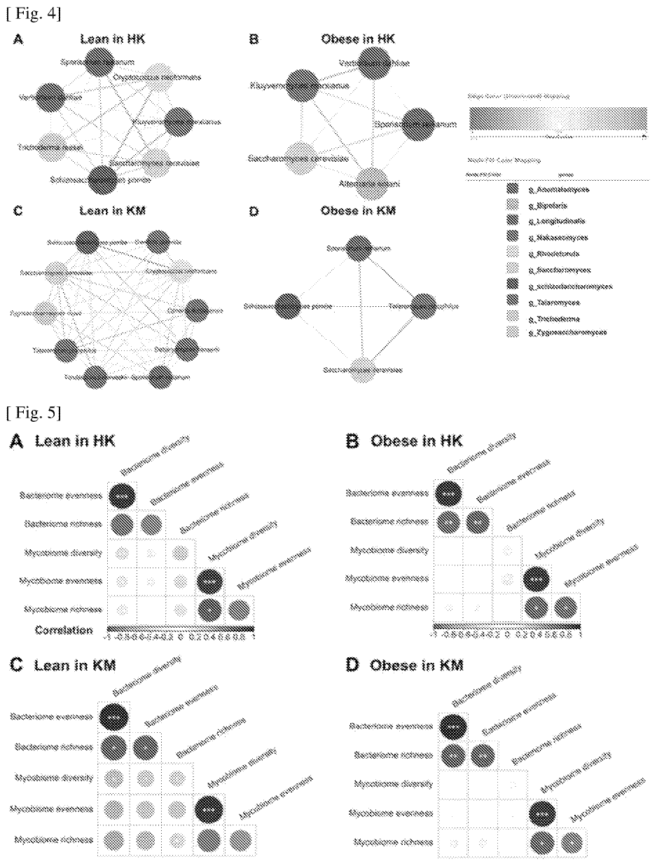

In the host, fungi interact with one another through hyphae or hitchhike to promote colonization in the gut. To study whether obesity influence fungal-fungal interactions, network analysis was performed to depict the relationship between the fungal species in two cohorts (FIG. 4).

In both lean groups from the Hong Kong (FIG. 4A) and Kunming (FIG. 4C) cohorts, more intricate intra-kingdom co-abundance relationships within the fecal mycobiome were observed when compared with co-abundance correlations in obesity (FIGS. 4B and D). Interestingly, a negative correlation between Sporisorium reilianum and Saccharomyces cerevisiae was identified across all four groups (Lean subjects in Hong Kong, r=−0.18, p<0.001; Obese subjects in Hong Kong, r=−0.08, p=0.14; Lean subjects in Kunming, r=−0.05, p=0.32; Obese subjects in Kunming, r=−0.13, p=0.32, FIG. 4), though not all significant, but might suggest an antagonistic relationship between these two species within human gut. In addition, a robust correlation between Schizosaccharomyces pombe and Cryptococcus neoformans was observed in both lean groups from Hong Kong (SparCC correlation, r=0.26, p<0.001) and Kunming (SparCC correlation, r=0.33, p<0.001) (FIGS. 4A and C), however, such association was not observed in their obese groups.

The fungal-fungal interaction networks between the lean and obese groups were also region-dependent (FIG. 4). Verticillium dahliae and Alternaria solani were uniquely identified in the co-occurrence network of the Hong Kong cohort, and A. solani was tightly associated with V. dahliae (r=0.15, p=0.01) and Kluyveromyces marxianus (SparCC correlation, r=−0.13, p<0.05) in the obese group, implying that A. solani might be a key participant in the gut of obese subjects in Hong Kong (FIGS. 4A and B). Meanwhile, the Kunming subjects were specifically distinguished from the Hong Kong subjects by Talaromyces pinophilus and Torulaspora delbruckli in the gut, and T. pinophilus was positively correlated with Candida dubliniensis (SparCC correlation, r=0.16, p<0.05) in the feces of lean subjects, however such symbiotic or synergistic relationship was clearly absent in the gut of obese individuals (FIGS. 4C and D).

Overall, a healthy gut mycobiome ecological network is featured by a complex gut fungal-fungal interactions, diverse fungal species compete or cooperate with each one in the host. In obese subjects, however, the ecological network became much less when there was no remarkable fungal diversity reduction shown in obesity. The decreased interactions were present with features depending on region.

### Decreased Fungal-Bacterial Inter-Kingdom Ecological Interactions in the Fecal Mycobiome in Obesity Across Regions

Previous study provides evidence that inter-kingdom interactions play a potential role in human metabolic health. The relationships between gut fungi and bacteria in feces were further explored by investigating correlations between the α diversity (diversity and richness) of mycobiome and bacteriome in obese subjects and lean controls.

As shown in FIG. 5, positive correlations between fungal-bacterial a diversity metrics were shown in both lean groups from the Hong Kong (FIG. 5A) and Kunming (FIG. 5C) cohorts. However, correlations of fungal-bacterial diversity were weak or even lost in the obese groups of Hong Kong (FIG. 5B, bacteriome diversity and mycobiome diversity, SparCC correlation, r=−0.007, p<0.01) and Kunming cohort (FIG. 5D, bacteriome diversity and mycobiome diversity, SparCC correlation, r=−0.01, p<0.01), compared to that in the lean groups of Hong Kong (FIG. 5A, bacteriome diversity and mycobiome diversity, SparCC correlation, r=0.23, p<0.01) and Kunming cohort (FIG. 5C, bacteriome diversity and mycobiome diversity, SparCC correlation, r=0.40, p<0.05), illustrating an altered fungal-bacterial network in obesity compared with that in lean controls. Furthermore, the correlations in the obese groups turned opposite to that observed in the lean groups (FIG. 5). For example, in Hong Kong, gut mycobiome evenness of lean subjects was positively associated with their bacteriome diversity (FIG. 5A, SparCC correlation, r=0.19, p<0.05), bacteriome evenness (FIG. 5A, r=0.13, p<0.05) and bacteriome richness (FIG. 5A, SparCC correlation, r=0.24, p=0.06), whereas significant negative correlations were present in the obese groups (FIG. 5B, SparCC correlation, r=−0.04, p<0.01; r=−0.06, p<0.01; r=−0.18, p<0.001, respectively).

Altogether, these results indicate a tight and synergetic interaction between fungal-bacterial inter-kingdom may contribute to maintaining a healthy ecological network of the gut microbiota in lean subjects, different from that in obesity.

Altered Gut Mycobiome Composition in Obese and Lean Subjects from Hong Kong and Kunming

To avoid geographic influence, fungal species associated with obesity were identified by LEfSe analysis comparing obese and lean individuals from Hong Kong and Kunming separately. The difference in relative abundance was confirmed by Wilcoxon rank-sum test. Relative abundance of Candida dubliniensis was significantly higher in lean subjects than obese subjects from Hong Kong (mean: 1.26% vs 0.66%, p<0.05, Wilcoxon rank-sum test) and Kunming (mean: 5.41% vs 0.83%, p<0.05, Wilcoxon rank-sum test).

In Hong Kong cohort, Cryptococcus neoformans and Schizosaccharomyces pombe were enriched in lean subjects (C. neoformans: mean relative abundance 2.41% vs 1.36%, p<0.05; S. pombe: mean relative abundance 3.99% vs 0.92%, p<0.05; Wilcoxon rank-sum test). Lachancea Thermotolerans, Saccharomyces paradox and Thielavia terrestris were enriched in obese subjects from Hong Kong (L. Thermotolerans: mean relative abundance 0.01% vs 0.05%, p<0.05; S. pombe: mean relative abundance 0.18% vs 0.26%, p<0.05; T. terrestris: mean relative abundance 0.57% vs 0.84%, Wilcoxon rank-sum test). In the Kunming cohort, Parastagonospora nodorum and Myceliophthora thermophila were associated with obesity (FIG. 2B).

Anti-Obesity Effect of Schizosaccharomyces pombe in High Fat Diet-Induced Obese Mice

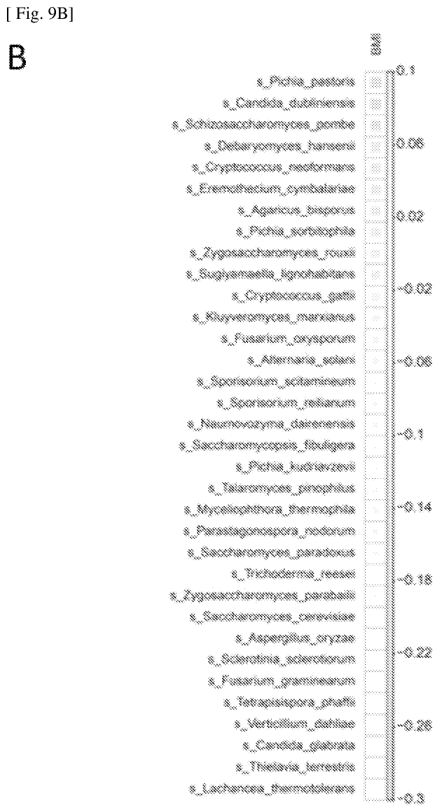

Among the differentially enriched fungal species identified by LEfSe analysis, the difference of S. pombe in the Hong Kong cohort was the most statistically significant (log relative abundance in lean vs obese: 3.99±7.49 vs 0.92±2.46, p<0.01, FIG. 2B). Further association investigation revealed its negative correlation with body mass index (BMI) in both Hong Kong cohort (r²=0.08, p=0.002, FIG. 9B). This indicates that S. pombe can protect against obesity, and potentially obesity-related condition such as diabetes and dyslipidemia.

To determine the causal effect of S. pombe on obesity, S. pombe (ATCC 16979) was concomitantly administered to mice that were fed with a high-fat diet (HF). In addition to PBS control, S. cerevisiae (ATCC 36375) was included as control (Group ND+SC and Group HF+SC, FIG. 6A). After 11 weeks of treatment, supplementation of HF with S. pombe but not S. cerevisae normalized diet-induced body weight gain. Body weight of mice treated with HF and S. pombe reduced by 20.86% compared to mice treated with HF and PBS (FIG. 6B, p<0.001), and by 13.87% compared to mice treated with HF and S. cerevisae (FIG. 6B, p<0.05). Interestingly, body weight of mice fed with ND supplemented with S. pombe remained similar to that fed with ND and PBS (p=0.101). This indicates S. pombe was well tolerated.

The risks associated with overweight or obesity are primarily related to the deposition of adipose tissue. While both subcutaneous and visceral adipose depots is associated with progressive deterioration in metabolic risk profile, visceral adiposity is more detrimental⁵. The effect of S. pombe on subcutaneous and visceral (epididymal and perirenal) adipose tissue was next evaluated. As expected, HF resulted in a marked increase in adipose weight relative to ND (Adipose mass index of HF vs ND, p<0.001, t-test; FIG. 6C). Oral supplementation of S. pombe but not S. cerevisae attenuated HDF-induced adiposity, as shown by the decreased subcutaneous (p<0.001; FIG. 6D) and perirenal (p<0.01; FIG. 6F) adipose tissue weight compared to mice fed with HF and PBS.

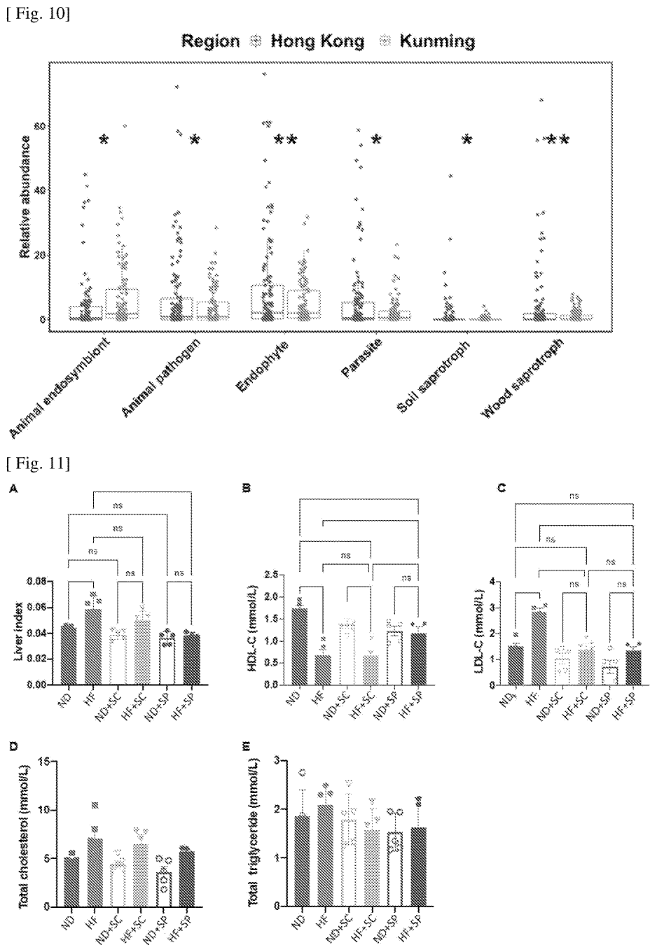

Dyslipidemia is a common metabolic disorder associated with obesity. In this study, it was observed that HF significantly increased serum level of low-density lipoprotein-cholesterol (LDL-C) (p<0.01) and decreased serum level of high-density lipoprotein-cholesterol (HDL-C) (p<0.001) compared to mice fed with ND (FIGS. 11B and C). Oral supplementation of S. pombe maintained serum LDL-C level similar to mice fed with ND (FIG. 11B). Serum HDL-C level was also significantly increased in mice fed with HF supplemented with S. pombe compared to mice fed with HF and PBS (p<0.05, FIG. 11C). As fatty liver is often associated with dyslipidemia and elevated LDL-C level was detected in HF-fed mice, lipid accumulation in liver tissue was next evaluated by assessing the liver mass index. It was observed that HF significantly increased liver mass index relative to ND-fed mice and oral supplementation with S. pombe protected HF-fed mice from such increase in liver mass index (p<0.001, FIG. 11A).

Non-alcoholic fatty liver disease (NAFLD) and type 2 diabetes mellitus (T2DM) are known to frequently coexist and act synergistically. The presence of NAFLD increases the risk of incident T2DM, with a relative risk of 1.86 (95% CI, 1.76-1.95) 6, while improvement of NAFLD is often associated with a reduced T2DM risk⁷. The effect of S. pombe on glucose tolerance and insulin resistance was therefore evaluated. Mice fed with HF developed glucose intolerance, as shown by a significant increase in the area under curve (AUC) derived from the oral glucose tolerance test (OGTT) compared to that of ND-fed mice (p<0.001, FIG. 6I-J). While supplementation with S. cerevisae did not result in any significant changes in the AUC of OGTT, supplementation with S. pombe reduced the AUC significantly (p<0.001, FIG. 6I-J). Insulin responsiveness was then assessed by insulin tolerance test (ITT). Mice fed with HF showed significantly increased AUC of ITT compared to mice fed with ND (p<0.001, FIG. 6G-H). While supplementation with S. cerevisae (p<0.05, FIG. 6G-H) resulted in the reduction of AUC of ITT, the effect of supplementation with S. pombe was much more significant (p<0.001, FIG. 6G-H).

Altogether, these results showed that supplementation of S. pombe improves HF-induced obesity, adiposity, dyslipidemia, fatty liver and T2DM (both glucose tolerance and insulin sensitivity). Importantly, S. pombe was well tolerated in both ND- and HF-fed mice. These findings provide support for the use of S. Pombe as a therapeutic option to target obesity and obesity-related condition such as adiposity, dyslipidemia, fatty liver and T2DM.

The use of this species, S. pombe (NCBI: txid4896), a fission yeast, is suitable for the prevention or amelioration as well as treatment of the above said diseases. This can be achieved by adjusting or modulating the level of S. pombe in the GI tract of a subject in need. Preferably the level of S. pombe should be increased to a relative abundance of 3.99%, which is the mean relative abundance in lean subjects from the Hong Kong cohort.

### Other Beneficial Species for Reducing Bodyweight

Comparing the mycobiome between lean and obese subjects from Hong Kong, it was discovered that in addition to S. pombe, Cryptococcus neoformans (NCBI: txid5207) was also enriched in lean subjects compared to obese subjects. Thus, in summary, to reduce bodyweight in a subject in need thereof, level of one or more of the fungal species shown in Table 4 in the GI tract may be adjusted or modulated by delivering to the subjects' GI tract an effective amount of these species. Preferably, the level of these species should be increased at least to the mean relative abundance in lean subjects (Table 4).

TABLE 4
Fungal species for reducing body weight

| | | | Relative Abundance in Lean Subjects (%) | | | |
|---|---|---|---|---|---|---|
| | | | | | Lower | Upper |
| Species | Cohort | NCBI:txid | Mean | Median | Quartile | Quartile |
| Cryptococcus | Hong | 5207 | 2.41 | 0.19 | 0 | 2.41 |
| neoformans | Kong | | | | | |
| Schizosaccharomyces | Hong | 4896 | 3.99 | 0.37 | 0 | 3.6 |
| pombe | Kong | | | | | |

### Administration of Fungal Species for Preventing or Ameliorating Obesity and Obesity-Related Condition or Maintaining Gut Health Against Pathogenic Candida

S. pombe, S. cerevisiae, D. hansenii and the fungal species listed in Table 4 can be obtained from a fungal culture in a sufficient quantity and then formulated into a suitable composition, to be introduced into the subject by oral, nasal, or rectal administration. The amount of each of the fungi or yeast in the composition is about 1×10⁶-1×10¹² CFU or 50 to 1000 mg. Ideally, the relative abundance should reach the median relative abundance (cut-off value), or within the range of lower and upper quartile listed in Table 4. Some of these species, although only present in low relative abundance (<0.005%), still play an important role in preventing or ameliorating obesity and obesity-related condition. For those species with a median relative abundance <0.005%, a detectable level (e.g., at least 0.005%) can be used as a cut-off value.

All patents, patent applications, and other publications, including GenBank Accession Numbers and equivalents, cited in this application are incorporated by reference in the entirety for all purposes.

### REFERENCES

1. Zuo T, Wong S H, Cheung C P, et al. Gut fungal dysbiosis correlates with reduced efficacy of fecal microbiota transplantation in Clostridium difficile infection. Nature communications 2018; 9:1-11.
2. Soverini M, Turroni S, Biagi E, et al. HumanMycobiomeScan: a new bioinformatics tool for the characterization of the fungal fraction in metagenomic samples. BMC genomics 2019; 20:496.
3. Truong D T, Franzosa E A, Tickle T L, et al. MetaPhlAn2 for enhanced metagenomic taxonomic profiling. Nature methods 2015; 12:902-903.
4. Nguyen N H, Song Z, Bates S T, et al. FUNGuild: an open annotation tool for parsing fungal community datasets by ecological guild. Fungal Ecology 2016; 20:241-248.
5. Cornier M A, Despres J P, Davis N, et al. Assessing adiposity: a scientific statement from the American Heart Association. Circulation 2011; 124:1996-2019.
6. Ballestri S, Zona S, Targher G, et al. Nonalcoholic fatty liver disease is associated with an almost twofold increased risk of incident type 2 diabetes and metabolic syndrome. Evidence from a systematic review and meta-analysis. J Gastroenterol Hepatol 2016; 31:936-44.
7. Targher G, Corey K E, Byrne C D, et al. The complex link between NAFLD and type 2 diabetes mellitus-mechanisms and treatments. Nat Rev Gastroenterol Hepatol 2021; 18:599-612.
8. Breuer U, Harms H. Debaryomyces hansenii—an extremophilic yeast with biotechnological potential. Yeast 2006; 23:415-437.
9. Capece A, Romano P. “Pecorino di Filiano” cheese as a selective habitat for the yeast species, Debaryomyces hansenii. International journal of food microbiology 2009; 132:180-184.
10. Ferreira A, Viljoen B. Yeasts as adjunct starters in matured Cheddar cheese. International journal of food microbiology 2003; 86:131-140.
11. Buzzini P, Martini A. Large-scale screening of selected Candida maltosa, Debaryomyces hansenii and Pichia anomala killer toxin activity against pathogenic yeasts. Sabouraudia 2001; 39:479-482.

1. A method for treating obesity in an obese subject or a subject at risk of obesity, comprising introducing into the gastrointestinal tract of the subject an effective amount of (1) one or more of Schizosaccharomyces pombe, Cryptococcus neoformans, and Saccharomyces cerevisae; or (2) an inhibitor of one or more of Lachancea thermotolerans, Saccharomyces paradoxus, Thielavia terrestris, Parastagonospora nodorum, and Myceliophthora thermophila.

2. The method of claim 1, comprising introducing into the gastrointestinal tract of the subject an effective amount of (1) one or more of Schizosaccharomyces pombe and Cryptococcus neoformans; or (2) an inhibitor of one or more of Lachancea thermotolerans, Saccharomyces paradoxus, and Thielavia terrestris.

3. The method of claim 1, wherein Schizosaccharomyces pombe, Cryptococcus neoformans, and/or Saccharomyces cerevisae level is increased by about 4-10 fold after the introducing step.

4. The method of claim 1, wherein Schizosaccharomyces pombe relative level is greater than about 3.99% of total gut fungi in the gastrointestinal tract of the subject after the introducing step.

5. The method of claim 1, wherein Lachancea thermotolerans, Saccharomyces paradoxus, Thielavia terrestris, Parastagonospora nodorum, and/or Myceliophthora thermophila relative level is less than about 0.1% of total gut fungi in the gastrointestinal tract of the subject after the introducing step.

6. The method of claim 1, wherein fungal alpha diversity index is increased by about 1.2 to 6 fold after the introducing step.

7. The method of claim 1, comprising introducing into the gastrointestinal tract of the subject a composition comprising:
(1) one or more of Schizosaccharomyces pombe, Cryptococcus neoformans, and Saccharomyces cerevisae; or (2) an inhibitor of one or more of Lachancea thermotolerans, Saccharomyces paradoxus, Thielavia terrestris, Parastagonospora nodorum, and Myceliophthora thermophila,
and a physiologically acceptable excipient.

8. The method of claim 7, wherein the composition comprises Schizosaccharomyces pombe and/or Cryptococcus neoformans.

9. The method of claim 7 or 8, wherein the composition is directly delivered to the subject's small intestine, ileum, or large intestine.

10. The method of claim 7 or 8, wherein the composition is formulated for oral ingestion.

11. The method of claim 10, wherein the composition is formulated in a daily dosage.

12. The method of claim 10, wherein the composition is in the form of a food or beverage item.

13. The method of claim 10, wherein the composition further comprises a prebiotic.

14. The method of any one of claims 1-13, wherein the introducing step is performed by fecal microbiota transplantation (FMT).

15. The method of claim 14, wherein the Schizosaccharomyces pombe, Cryptococcus neoformans, and/or Saccharomyces cerevisae is derived from a donor's fecal material.

16. The method of any one of claims 1-15, wherein the subject's bodyweight is reduced, serum low-density lipoprotein-cholesterol (LDL-C) level is reduced, serum glucose level is reduced, glucose tolerance is increased, and/or insulin responsiveness is increased.

17. A kit for promoting weight loss or reducing risk of obesity in a subject comprising a plurality of compositions each comprising an effective amount of one or more of the following: Schizosaccharomyces pombe; Cryptococcus neoformans; Saccharomyces cerevisae; an inhibitor of Lachancea thermotolerans; an inhibitor of Saccharomyces paradoxus; an inhibitor of Thielavia terrestris; an inhibitor of Parastagonospora nodorum, and an inhibitor of Myceliophthora thermophila.

18. The kit of claim 17, comprising a first composition comprising effective amount of Schizosaccharomyces pombe and/or Cryptococcus neoformans, and a second composition comprising an inhibitor of Lachancea thermotolerans, Saccharomyces paradoxus, and/or Thielavia terrestris.

19. The kit of claim 17, comprising a first composition comprising an effective amount of Schizosaccharomyces pombe, and a second composition comprising an effective amount of Cryptococcus neoformans.

20. The kit of claim 18 or 19, wherein the compositions are formulated for oral administration or for direct delivery to the subject's gastrointestinal tract.
